(19) United States
(12) Patent Application Publication
(10) Pub. No.: US 2024/0138763 A1
(43) Pub. Date: May 2, 2024

(54) ASSISTED BRACKET FOR POSTOPERATIVE JOINT ACTIVITY EXERCISE

(71) Applicant: HAN XIE, Xiaogan (CN)

(72) Inventor: HAN XIE, Xiaogan (CN)

(21) Appl. No.: 18/407,577

(22) Filed: Jan. 9, 2024

Related U.S. Application Data
(63) Continuation of application No. PCT/CN2023/107523, filed on Jul. 14, 2023.

(30) Foreign Application Priority Data
Jul. 18, 2022 (CN) 202210839387.4

(51) Int. Cl. A61B 5/00; A61B 5/11; A61F 5/01

(52) U.S. Cl. CPC A61B 5/6812; A61B 5/1121; A61F 5/0102; A61B 2505/09

(57) ABSTRACT

The assisted bracket for postoperative joint activity exercise is provided, including an inner shell bracket, a primary swing arm bracket, a secondary swing arm bracket, an angle sensor, and a shell body. By setting up an assisted bracket for postoperative joint activity exercise, the primary swing arm bracket is positioned on the proximal limb of the patient, and the secondary swing arm bracket is positioned on the distal limb of the patient. The angle sensor is connected to the mobile APP in electrical signals through a communication module to facilitate patient movement, it can drive the primary and secondary swing arm brackets to move, allowing the angle sensor to record their motion data, thereby monitoring the exercise amount and exercise frequency of the patient. This allows patients to better engage in rehabilitation exercises and improve the effectiveness of postoperative rehabilitation exercises.

### CROSS-REFERENCE TO RELATED APPLICATIONS

This application is a continuation of International Application No. PCT/CN2023/107523, filed on Jul. 14, 2023, which claims foreign priority of Chinese Patent Application No. 202210839387.4, filed on Jul. 18, 2022 in the State Intellectual Property Office of China, the contents of all of which are hereby incorporated by reference.

### TECHNICAL FIELD

The present disclosure relates to the technical field of medical auxiliary devices, in particular to an assisted bracket for postoperative joint activity exercise.

### BACKGROUND

The connection between bones is called a joint, and after joint surgery, doctors usually encourage patients to engage in appropriate exercise to assist in promoting joint recovery.

But patients often experience poor medical compliance due to pain in joint wounds after surgery, resulting in reluctance or complete inactivity, which affects the speed and effectiveness of joint recovery after surgery.

However, a single medical staff needs to be responsible for multiple patients, making it difficult for them to constantly supervise each patient, which is not conducive to patient recovery. Thus, the present disclosure proposes an assisted bracket for postoperative joint activity exercise to solve the above problem.

### SUMMARY

The objective of the present disclosure is to provide an assisted bracket for postoperative joint activity exercise to solve the problems mentioned in the background.

To achieve the above objective, the present disclosure provides the following technical solution: An assisted bracket for postoperative joint activity exercise, which includes:
an inner shell bracket, wherein the inner shell bracket is provided with a primary installation hole on a bottom surface, the bottom surface of the inner shell bracket is integrally formed with a lap, and an outer surface of the inner shell bracket is provided with a sensor clamping slot at an edge position of the primary installation hole;
a primary swing arm bracket, wherein the primary swing arm bracket is fixedly connected to the inner shell bracket, and the primary swing arm bracket is provided with a primary rotating hole and a guide hole;
a secondary swing arm bracket, wherein the secondary swing arm bracket is provided with a secondary rotating hole and a secondary installation hole, and the secondary swing arm bracket is rotatably connected to the primary swing arm bracket through a primary fastener;
an angle sensor embedded in the sensor clamping slot installed in the inner shell bracket; and
the shell body clamped and installed on the inner shell bracket.

Preferably, the primary installation hole, the primary rotating hole, and the secondary rotating hole are correspondingly arranged; the primary fastener is composed of a combination of a limit cap, a limit rod, an installation part, and a limit part, and the limit rod is tightened with a limit buckle.

Preferably, there is a gap between the limit buckle and the secondary swing arm bracket when the limit buckle is actually tightened.

Preferably, the guide hole is a semi-circular groove, the secondary installation hole is arranged corresponding to the guide hole, a distance between the secondary installation hole and the guide hole is limited by a secondary fastener and a limit nut, and there is a gap between the limit nut and the secondary swing arm bracket when the limit nut is actually tightened.

Preferably, the lap is integrally formed with the inner shell bracket, the primary swing arm bracket is installed on the lap, a front end of the inner shell bracket is integrally formed with a clamping protrusion, and a front end of the primary swing arm bracket is clamped and positioned through the clamping protrusion.

Preferably, the installation part is a rectangular seat structure, an installation groove on a rotation center axis of the angle sensor corresponds to a cross-sectional size of the installation part, a limited protrusion is set on a side of the limit part, and the angle sensor is clamped and positioned on the limit part through the limited protrusion.

Preferably, the angle sensor is connected to a communication module through electrical signals, and the communication module is connected to a mobile APP through electrical signals.

Preferably, the communication module at least comprises a Bluetooth module, a wired network communication module, and a wireless network communication module.

Preferably, the primary swing arm bracket is provided with a primary bandage slot, the secondary swing arm bracket is provided with a secondary bandage slot, the primary bandage slot and the secondary bandage slot are respectively connected with a primary bandage and a secondary bandage, the primary swing arm bracket is positioned on a proximal limb of the patient through the primary bandage, and the secondary swing arm bracket is positioned on a distal limb of the patient through the secondary bandage.

Preferably, the primary bandage slot and the secondary bandage slot are respectively arranged in a row with equal spacing.

Compared with the prior art, the advantageous effects of the present disclosure are as below:

By setting up an assisted bracket for postoperative joint activity exercise consisting of an inner shell bracket, a primary swing arm bracket, a secondary swing arm bracket, an angle sensor, and a shell body, the primary swing arm bracket is positioned on the proximal limb of the patient, and the secondary swing arm bracket is positioned on the distal limb of the patient. The angle sensor is connected to the mobile APP in electrical signals through a communication module to facilitate patient movement, it can drive the primary and secondary swing arm brackets to move, allowing the angle sensor to record their motion data, thereby monitoring the exercise amount and exercise frequency of the patient. This allows patients to better engage in rehabilitation exercises and improve the effectiveness of postoperative rehabilitation exercises.

### BRIEF DESCRIPTION OF DRAWINGS

FIG. 1 is a schematic diagram of the structure of the present disclosure;

FIG. 2 is a schematic diagram of the installation position of the angle sensor of the present disclosure;

FIG. 3 is a schematic diagram of the connection between the primary swing arm bracket and the secondary swing arm bracket of the present disclosure;

FIG. 4 is a schematic diagram of the outer side of the inner shell bracket of the present disclosure;

FIG. 5 is a schematic diagram of the inner side of the inner shell bracket of the present disclosure;

FIG. 6 is an enlarged partial view of the structure at position A in FIG. 5;

FIG. 7 is a schematic diagram of the structure of the primary swing arm bracket;

FIG. 8 is a schematic diagram of the structure of the secondary swing arm bracket;

FIG. 9 is a schematic diagram of the structure of the primary fastener;

FIG. 10 is a schematic diagram of the limit buckle; and

FIG. 11 is a schematic diagram of the circuit connection of the angle sensor of the present disclosure.

Reference number in the figure: 1—inner shell bracket, 2—primary swing arm bracket, 3—secondary swing arm bracket, 4—angle sensor, 5—shell body, 6—primary fastener, 7—limit buckle, 8—secondary fastener, 9—limit nut, 10—primary installation hole, 11—lap, 12—secondary rotating hole, 13—guide hole, 14—secondary installation hole, 15—limit cap, 16—limit rod, 17—installation part, 18—limit part, 19—clamping protrusion, 20—sensor clamping slot, 21—primary bandage slot, 22—secondary bandage slot.

### DESCRIPTION OF EMBODIMENTS

The following will provide a clear and complete description of the technical solution in the embodiments of the present disclosure, in conjunction with the accompanying drawings. It is apparent that the described embodiments are only a part of the embodiments of the present disclosure, not all of them. Based on the embodiments in the present disclosure, all other embodiments obtained by ordinary skilled person in the art without creative labor fall within the scope of the present disclosure.

In order to provide a clear and complete description of the objective, technical solution, and advantages of the present disclosure, the following will provide further detailed illustration of the embodiments of the present disclosure in conjunction with the accompanying drawings. It should be understood that the specific embodiments described here are part of the embodiments of the present disclosure, not all of them. They are only used to explain the embodiments of the present disclosure and are not intended to limit them. All other embodiments obtained by ordinary skilled person in the art without creative labor fall within the scope of the present disclosure.

In the description of the present disclosure, it should be noted that the terms “center”, “middle”, “up”, “down”, “left”, “right”, “inside”, “outside”, “top”, “bottom”, “side”, “vertical”, “horizontal”, etc. indicate an orientation or positional relationship based on the orientation or positional relationship shown in the accompanying drawings, merely for the convenience of describing the present disclosure and simplifying the description, rather than indicating or implying that the device or component referred to must have a specific orientation, be constructed and operated in a specific orientation, therefore it cannot be understood as a limitation of the present disclosure. In addition, the terms “one”, “first”, “second”, “third”, “fourth”, “fifth”, “sixth” are only used for description and cannot be understood as indicating or implying relative importance.

In the description of the present disclosure, it should be noted that unless otherwise specified and limited, the terms “installation”, “connection”, and “connected” should be understood broadly, for example, it may be fixed connections, detachable connections, or integrated connections; it may be a mechanical connection or an electrical connection; it may be directly connected, or indirectly connected through an intermediate medium, or it may be the internal connection between two components. For ordinary skilled person in the art, the specific meanings of the above terms in the present disclosure can be understood in specific circumstances.

For the purpose of simplicity and illustration, the principles of the embodiments are mainly described by reference to examples. In the following description, many specific details are provide a thorough understanding of the embodiments. However, it is evident that for ordinary skilled person in the art, these embodiments may not be limited to these specific details in practice. In some embodiments, there is no detailed description of the well-known methods and structures to avoid unnecessarily making these embodiments difficult to understand. Additionally, all embodiments can be combined with each other for use.

### Embodiment 1

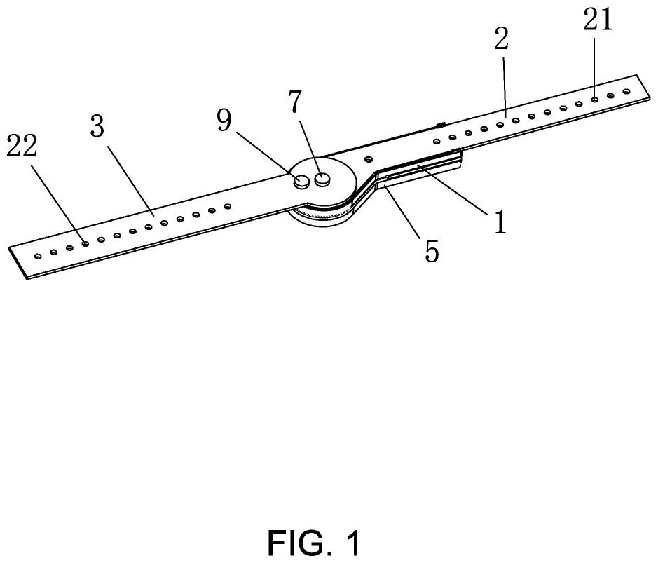

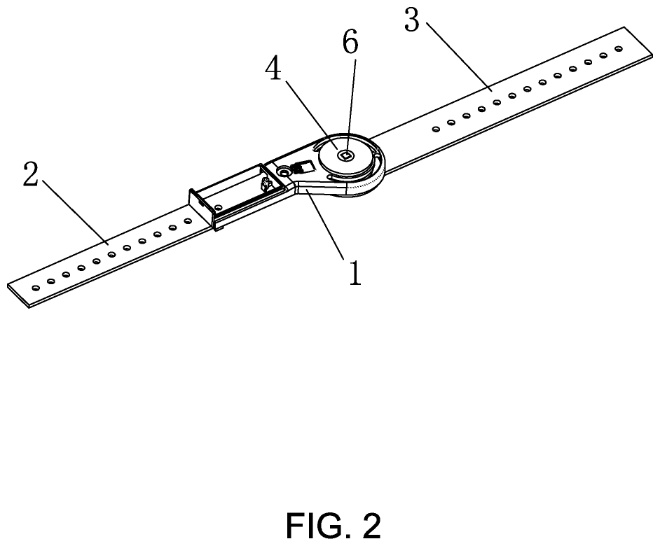

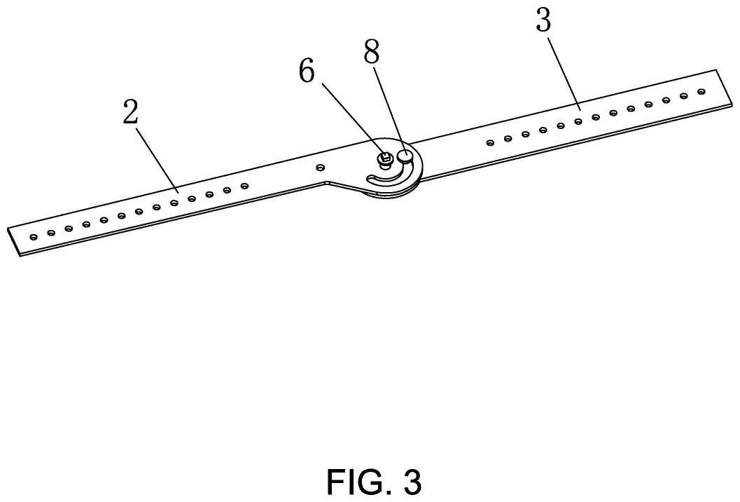

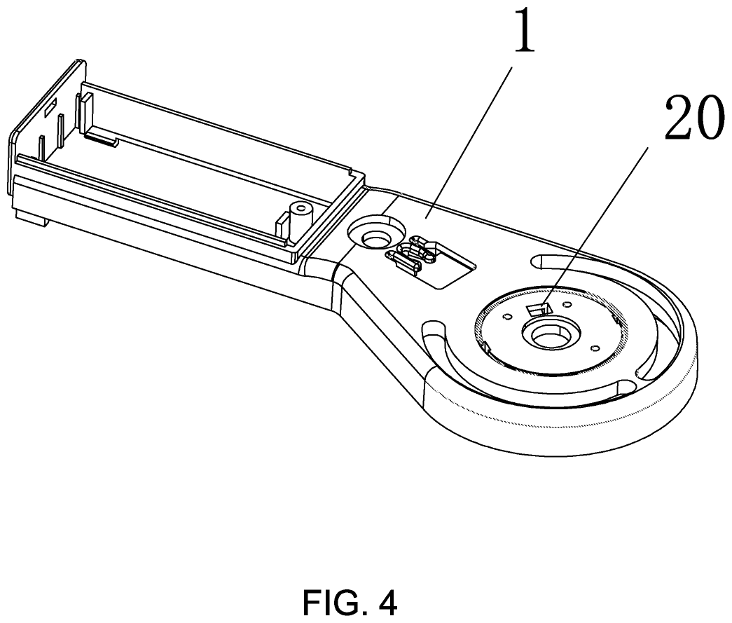

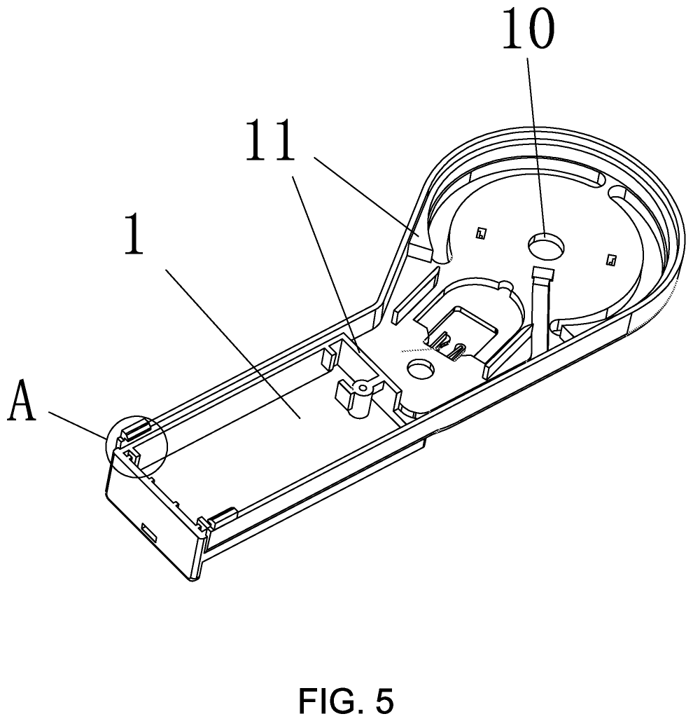

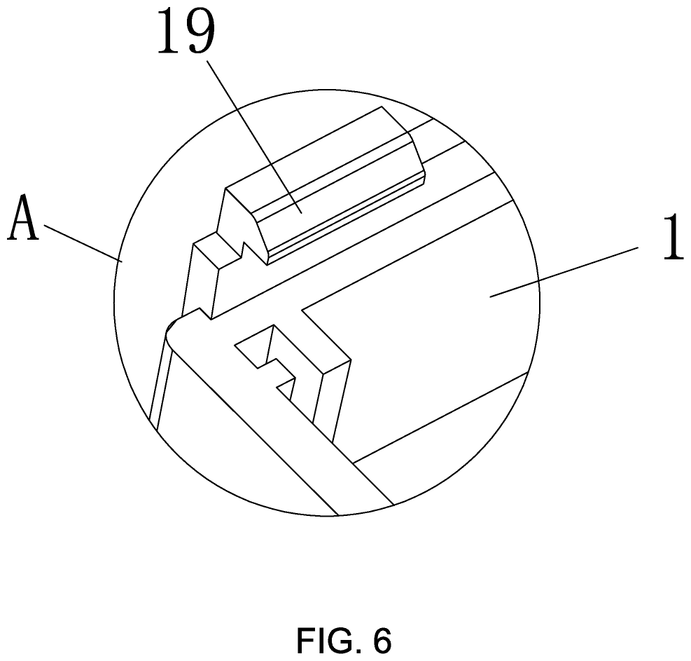

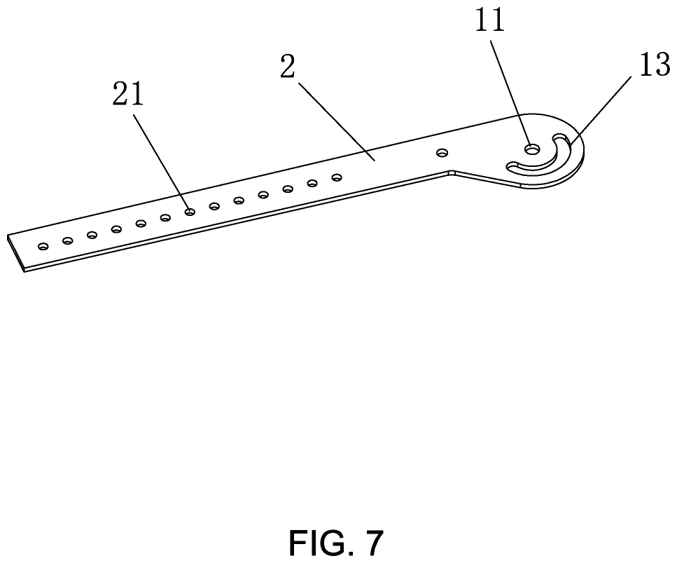

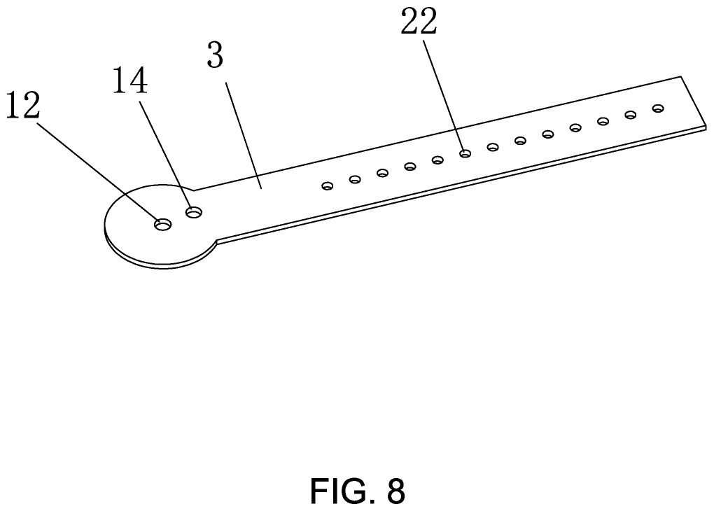

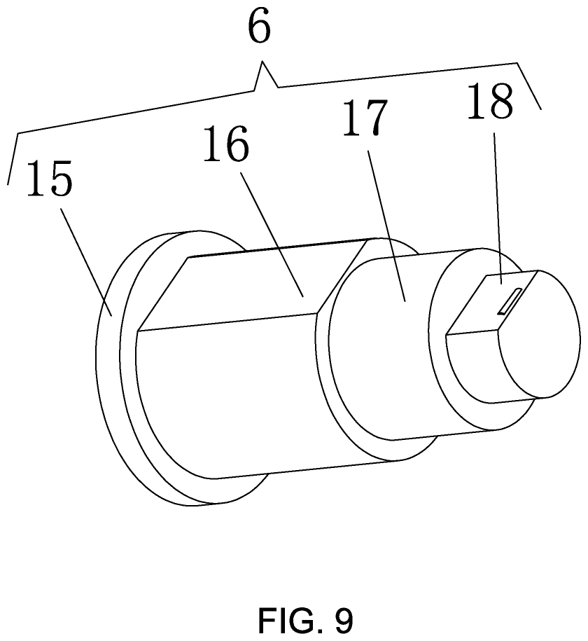

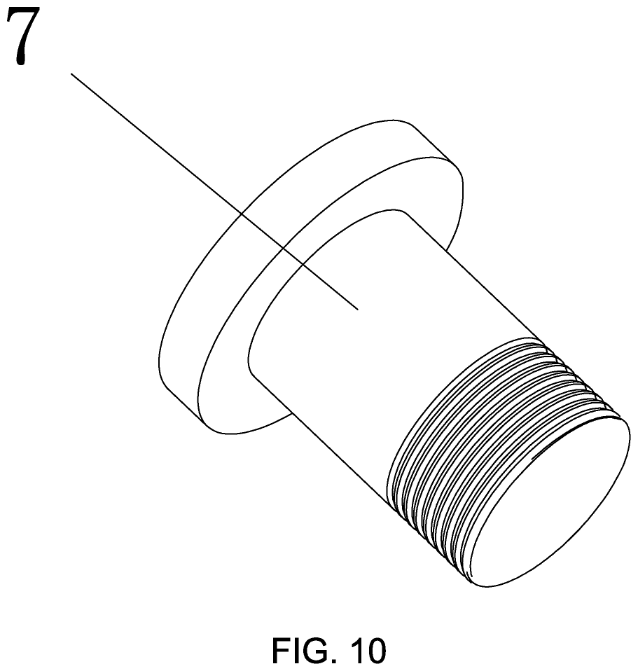

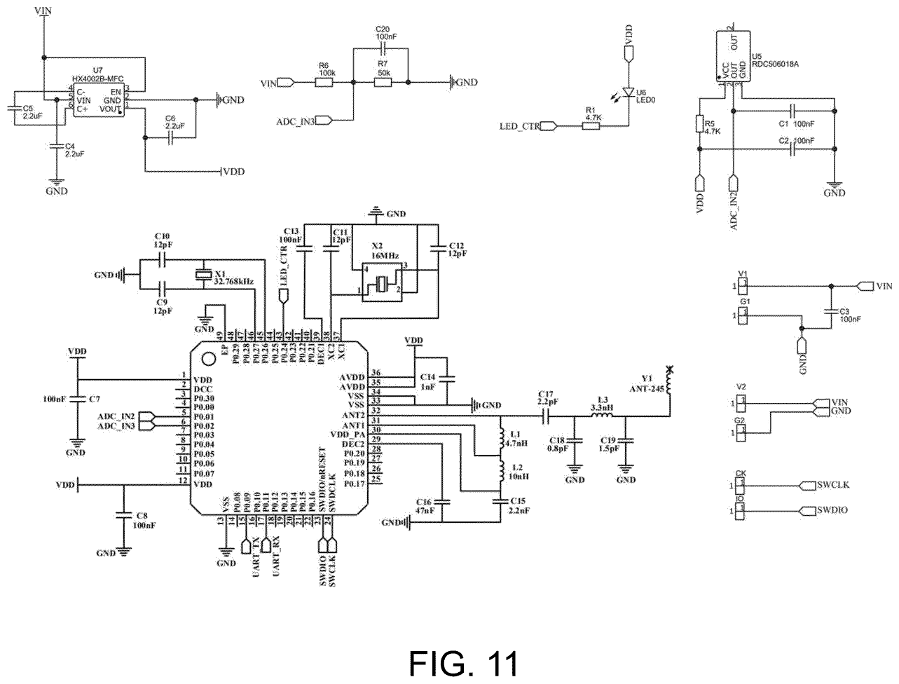

Please refer to FIGS. 1-11, an assisted bracket for postoperative joint activity exercise is provided by the present disclosure, which includes an inner shell bracket 1, a primary swing arm bracket 2, a secondary swing arm bracket 3, an angle sensor 4, and the shell body 5. The inner shell bracket 1 is provided with a primary installation hole 10 on the bottom surface, the bottom surface of the inner shell bracket 1 is integrally formed with a lap 11, and the outer surface of the inner shell bracket 1 is provided with a sensor clamping slot 20 at the edge position of the primary installation hole 10. The primary swing arm bracket 2 is fixedly connected to the inner shell bracket 1, and the primary swing arm bracket 2 is provided with a primary rotating hole 11 and a guide hole 13. The secondary swing arm bracket is provided with a secondary rotating hole 12 and a secondary installation hole 14, and the secondary swing arm bracket 3 is rotatably connected to the primary swing arm bracket 2 through a primary fastener 6. The angle sensor 4 is embedded in the sensor clamping slot 20 installed in the inner shell bracket 1. The shell body 5 is clamped and installed on the inner shell bracket 1.

The primary installation hole 10, the primary rotating hole 11, and the secondary rotating hole 12 are correspondingly arranged. The primary fastener 6 is composed of a combination of a limit cap 15, a limit rod 16, an installation part 17, and a limit part 18, and the limit rod 16 is tightened with a limit buckle 7.

There is a gap between the limit buckle 7 and the secondary swing arm bracket 3 when the limit buckle 7 is actually tightened.

The guide hole 13 is a semi-circular groove, the secondary installation hole 14 is arranged corresponding to the guide hole 13. The distance between the secondary installation hole 14 and the guide hole 13 is limited by a secondary fastener 8 and a limit nut 9, and there is a gap between the limit nut 9 and the secondary swing arm bracket 3 when the limit nut 9 is actually tightened.

The lap 11 is integrally formed with the inner shell bracket 1, the primary swing arm bracket 2 is installed on the lap 11, the front end of the inner shell bracket 1 is integrally formed with a clamping protrusion 19, and the front end of the primary swing arm bracket 2 is clamped and positioned through the clamping protrusion 19.

The installation part 17 is a rectangular seat structure, and an installation groove on the rotation center axis of the angle sensor 4 corresponds to the cross-sectional size of the installation part 17. A limited protrusion is set on the side of the limit part 18, and the angle sensor 4 is clamped and positioned on the limit part 18 through the limited protrusion.

The angle sensor 4 is connected to a communication module through electrical signals.

The communication module at least includes a Bluetooth module, a wired network communication module, and a wireless network communication module. The communication module is connected to a mobile APP through electrical signals. By setting up an assisted bracket for postoperative joint activity exercise consisting of the inner shell bracket 1, the primary swing arm bracket 2, the secondary swing arm bracket 3, the angle sensor 4, and the shell body 5, the primary swing arm bracket 2 is positioned on the proximal limb of the patient, and the secondary swing arm bracket 3 is positioned on the distal limb of the patient. The angle sensor 4 is connected to the mobile APP in electrical signals through a communication module to facilitate patient movement, it can drive the primary swing arm bracket 2 and secondary swing arm bracket 3 to move, allowing the angle sensor 4 to record their motion data, thereby monitoring the exercise amount and exercise frequency of the patient. This allows patients to better engage in rehabilitation exercises and improve the effectiveness of postoperative rehabilitation exercises.

### Embodiment 2

On the basis of Embodiment 1, the primary swing arm bracket 2 is provided with a primary bandage slot 21, the secondary swing arm bracket 3 is provided with a secondary bandage slot 22, the primary bandage slot 21 and the secondary bandage slot 22 are respectively connected with a primary bandage and a secondary bandage, the primary swing arm bracket 2 is positioned on the proximal limb of the patient through the primary bandage, and the secondary swing arm bracket 3 is positioned on the distal limb of the patient through the secondary bandage. The primary bandage slot 21 and the secondary bandage slot 22 are respectively arranged in a row with equal spacing, making it easy to adjust the bandage position of the bandage according to different patients.

Although the specific embodiments of the present disclosure have been described above to facilitate understanding by those skilled in the art, this disclosure is not limited to the specific embodiments. For ordinary skilled in the art, as long as various variations are within the spirit and scope of the present disclosure as defined and determined by the attached claims, all disclosure creations utilizing the concept of the present disclosure are protected.

What is claimed is:

1. An assisted bracket for postoperative joint activity exercise, comprising:
an inner shell bracket (1), wherein the inner shell bracket (1) is provided with a primary installation hole (10) on a bottom surface, the bottom surface of the inner shell bracket (1) is integrally formed with a lap (11), and an outer surface of the inner shell bracket (1) is provided with a sensor clamping slot (20) at an edge position of the primary installation hole (10);
a primary swing arm bracket (2), wherein the primary swing arm bracket (2) is fixedly connected to the inner shell bracket (1), and the primary swing arm bracket (2) is provided with a primary rotating hole (11) and a guide hole (13);
a secondary swing arm bracket (3), wherein the secondary swing arm bracket is provided with a secondary rotating hole (12) and a secondary installation hole (14), and the secondary swing arm bracket (3) is rotatably connected to the primary swing arm bracket (2) through a primary fastener (6);
an angle sensor (4) embedded in the sensor clamping slot (20) installed in the inner shell bracket (1); and
the shell body (5) clamped and installed on the inner shell bracket (1).

2. The assisted bracket for postoperative joint activity exercise according to claim 1, wherein the primary installation hole (10), the primary rotating hole (11), and the secondary rotating hole (12) are correspondingly arranged; the primary fastener (6) is composed of a combination of a limit cap (15), a limit rod (16), an installation part (17), and a limit part (18), and the limit rod (16) is tightened with a limit buckle (7).

3. The assisted bracket for postoperative joint activity exercise according to claim 2, wherein there is a gap between the limit buckle (7) and the secondary swing arm bracket (3) when the limit buckle (7) is actually tightened.

4. The assisted bracket for postoperative joint activity exercise according to claim 3, wherein the guide hole (13) is a semi-circular groove, the secondary installation hole (14) is arranged corresponding to the guide hole (13), a distance between the secondary installation hole (14) and the guide hole (13) is limited by a secondary fastener (8) and a limit nut (9), and there is a gap between the limit nut (9) and the secondary swing arm bracket (3) when the limit nut (9) is actually tightened.

5. The assisted bracket for postoperative joint activity exercise according to claim 4, wherein the lap (11) is integrally formed with the inner shell bracket (1), the primary swing arm bracket (2) is installed on the lap (11), a front end of the inner shell bracket (1) is integrally formed with a clamping protrusion (19), and a front end of the primary swing arm bracket (2) is clamped and positioned through the clamping protrusion (19).

6. The assisted bracket for postoperative joint activity exercise according to claim 5, wherein the installation part (17) is a rectangular seat structure, an installation groove on a rotation center axis of the angle sensor (4) corresponds to a cross-sectional size of the installation part (17), a limited protrusion is set on a side of the limit part (18), and the angle sensor (4) is clamped and positioned on the limit part (18) through the limited protrusion.

7. The assisted bracket for postoperative joint activity exercise according to claim 6, wherein the angle sensor (4) is connected to a communication module through electrical signals, and the communication module is connected to a mobile APP through electrical signals.

8. The assisted bracket for postoperative joint activity exercise according to claim 7, wherein the communication module at least comprises a Bluetooth module, a wired network communication module, and a wireless network communication module.

9. The assisted bracket for postoperative joint activity exercise according to claim 6, wherein the primary swing arm bracket (2) is provided with a primary bandage slot (21), the secondary swing arm bracket (3) is provided with a secondary bandage slot (22), the primary bandage slot (21) and the secondary bandage slot (22) are respectively connected with a primary bandage and a secondary bandage, the primary swing arm bracket (2) is positioned on a proximal limb of the patient through the primary bandage, and the secondary swing arm bracket (3) is positioned on a distal limb of the patient through the secondary bandage.

10. The assisted bracket for postoperative joint activity exercise according to claim 8, wherein the primary bandage slot (21) and the secondary bandage slot (22) are respectively arranged in a row with equal spacing.
